(19) United States
(12) Patent Application Publication
(10) Pub. No.: US 2022/0202870 A1
(43) Pub. Date: Jun. 30, 2022

(54) COMPOSITION

(71) Applicant: AGENCY FOR SCIENCE, TECHNOLOGY AND RESEARCH, Singapore (SG)

(72) Inventor: Sai Kiang LIM, Singapore (SG)

(73) Assignee: AGENCY FOR SCIENCE, TECHNOLOGY AND RESEARCH, Singapore (SG)

(21) Appl. No.: 17/604,526

(22) PCT Filed: Apr. 17, 2020
(86) PCT No.: PCT/SG2020/050243

§ 371 Date: Oct. 18, 2021

(30) Foreign Application Priority Data
Apr. 18, 2019 (SG) 10201903524W

(51) Int. Cl. A61K 35/28; A61K 9/107; A61K 9/127; A61K 47/24; A61K 9/00; A61P 17/06

(52) U.S. Cl. CPC A61K 35/28; A61K 9/107; A61P 17/06; A61K 47/24; A61K 9/0014; A61K 9/127

(57) ABSTRACT

The invention relates to a pharmaceutical composition and the method of preparing the pharmaceutical composition, wherein the composition comprises an oil-in-water emulsion of an exosome, such as a mesenchymal stem cell (MSC) exosome, for the treatment or prevention of a dermatological disease or condition, such as psoriasis.

### FIELD

This invention relates to the fields of medicine, cell biology, molecular biology and genetics.

### BACKGROUND

Topical application of drugs has many advantages (reviewed⁸). It could potentially bypass several issues associated with oral or intravenous administration such as first-pass metabolism resulting in higher bioavailability and reduced administration frequency, reduced side effects and poor patient compliance.

However, overcoming the stratum corneum of our skin to deliver drugs has been challenging. To date, only a few small (<500 Da), neutral, lipophilic molecules could be delivered by topical application. Hence, it is generally recognized that any transdermal drug delivery would require physical forces such as microneedles⁴, iontophoresis⁵ or sonophoresis⁶ to first break down or penetrate the stratum corneum.

Lipid vesicle carriers⁷ such as liposomes were also proposed as potential transdermal delivery vehicles. Liposomes are widely used in the cosmetic industry and purportedly transport and deliver drugs through the skin barrier^{9,10}. However, advance imaging technologies such as scanning electron microscopy (SEM) and transmission electron microscopy (TEM), laser scanning confocal (LSM), multi photon excitation fluorescence microscopy (MPEM), a combination of cross correlation raster image correlations spectroscopy (CC-RICS) and finally Superresolution and Fluorescence Dynamics have demonstrated that liposomes burst in the stratum corneum and do not carry their drug load beyond the stratum corneum^{7,9-14}.

Biological materials that are used to alleviate specific immune molecules/complexes in the treatment of dermatological diseases are generally administered either intravenously or subcutaneously.

This is because unlike open wounds or burns where the skin barriers are highly compromised, most dermatological disorders or diseases such as psoriasis or atopic dermatitis have largely intact skin barriers. As such, topical application of relatively large water soluble biological materials will not be therapeutically efficacious as they cannot cross the skin barrier to modulate immune activity. They would likely require a permeability enhancer such as microneedles⁴, iontophoresis⁵, sonophoresis⁶, and lipid vesicle carriers⁷.

With the development of biological materials as dermatological therapeutics, there is an increasing need to deliver these materials topically especially for skin disorders. Topical application of such biological materials however remains challenging.

### SUMMARY

We disclose a topical formulation to facilitate the immune modulating activities of biological materials such as exosomes on dermatological disease lesions caused by psoriasis or atopic dermatitis.

We further disclose the efficacy of topically applied MSC exosomes in alleviating dermatologically important immune molecules or complexes, IL-17, IL-23, TNFα, and Membrane Attack Complex C5b-9.

According to a 1^{st} aspect of the present invention, we provide a pharmaceutical composition for the treatment or prevention of a dermatological disease or condition. The pharmaceutical composition may comprise an oil-in-water emulsion of an exosome. The exosome may comprise a mesenchymal stem cell (MSC) exosome.

The dermatological disease or condition may comprise atopic dermatitis, psoriasis or eczema.

The pharmaceutical composition may be capable of reducing or alleviating one or more of erythema, scaling and thickness in an individual. The reduction may be as compared to an individual to which the pharmaceutical composition is not applied

The pharmaceutical composition may be capable of reducing the level of an inflammatory cytokine such as TNFα, IL-17, IL-23 and Membrane Attack Complex C5b-9. The reduction may be as compared to an individual to which the pharmaceutical composition is not applied.

The pharmaceutical composition may a lotion. The pharmaceutical composition may be topically applied to a skin of an individual in need of treatment.

The pharmaceutical composition may be administered 2 to 3 times daily.

The pharmaceutical composition may alleviate immune reactivity triggered by exposure of the skin of an individual to an irritant. The irritant may comprise Imiquimod (IMQ).

The pharmaceutical composition may further comprise a liposome comprising a lecithin. The lecithin may comprise egg-yolk lecithin. The liposome comprising a lecithin may comprise a yolk lecithin liposome (YLL).

There is provided, according to a 2^{nd} aspect of the present invention, use of an oil-in-water emulsion of an exosome in the preparation of a medicament for the treatment or prevention of a dermatological disease or condition. The exosome may comprise a mesenchymal stem cell (MSC) exosome.

The use may comprise any feature as set out above for the 1^{st} aspect of the invention.

We provide, according to a 3^{rd} aspect of the present invention, a method of preparing a pharmaceutical composition for the treatment or prevention of a dermatological condition

The method may comprise providing an exosome such as an mesenchymal stem cell exosome (MSC). The method may comprise homogenising the exosome with an emulsifying agent to form an oil in water emulsion of the exosome.

The mesenchymal stem cell exosome (MSC) may be provided as a dispersion in an oil. The oil may comprise a plant oil. The oil may comprise olive oil.

The preparation of the pharmaceutical composition may comprise a period of storage of the MSC dispersion in oil prior to the homogenising step.

The oil-in-water emulsion of an exosome may be prepared by homogenising the MSC with an emulsifying agent.

The emulsifying agent may be selected from the group consisting of a natural emulsifying agent, agar, tragacanth, alginates, gum, pectins, acacia, starch, gelatin, albumin, lecithin and cholesterol.

The emulsifying agent may be selected from the group consisting of a semi-synthetic emulsifying agent, methylcellulose derivatives such as carboxymethylcellulose and starch derivatives such as octenyl succinate anhydride treated starch, also known as OSA-starch.

The emulsifying agent may be selected from the group consisting of a synthetic emulsifying agent, benzalkonium chloride, benzethonium chloride, sodium oleate potassium oleate, triethanolamine stearate, detergents such as sodium lauryl sulfate, sodium dioctyl sulfosuccinate, sodium docusate), sorbitan esters (Spans) and polyoxyethylene derivatives of sorbitan esters (Tweens) and glyceryl esters.

The emulsifying agent may comprises one or more, such as all of polyacrylate-13, polyisobutene and polysorbate 20.

The emulsifying agent may comprise a 2% (w/v) solution of SEPIPLUS™ 400 in water.

The preparation may further comprise a step of adding a liposome comprising a lecithin to the pharmaceutical composition. The lecithin may comprise egg-yolk lecithin. The liposome comprising a lecithin may comprise a yolk lecithin liposome (YLL).

As a 4^{th} aspect of the present invention, there is provided a pharmaceutical composition prepared by a method according to the 3^{rd} aspect of the invention.

The pharmaceutical composition as prepared may comprise any feature as set out above for the 1^{st} aspect of the invention.

The practice of this invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-314-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

### BRIEF DESCRIPTION OF THE FIGURES

FIG. 1 is a drawing showing human skin biopsy samples treated with 50 ul of PBS or fluorescence-labelled exosome for 2 hours at 37° C. incubator. Thereafter, the skin biopsies were gently washed with PBS and placed in OCT quick-freeze medium compound. Samples were either frozen immediately or after an 8 hour-incubation in OCT medium using liquid nitrogen. 3 um sections were made and then subjected to Haematoxylin and Eosin staining (H&E) analysis and green fluorescence screening. Images were taken using EVOS microscope for H&E and Carl Zeiss microscope for fluorescence (Dapi and FL 488 detection).

FIG. 2 is a drawing showing lyophilized exosome resuspended in olive oil and kept at 40° C. for one week. The suspension was extracted with water and assayed for CD73 activity. The activity was measured against the CD73 activity in an equivalent amount of lyophilized exosome. The CD73 level was 20% lower, presumably from the loss in extraction and heat. Therefore, if kept at room temperature of ˜30° C., the lyophilized exosome is likely to be stable.

FIG. 3 is a drawing showing a summary of Experiment 1 and Experiment 2.

FIG. 4 is a drawing showing skin phenotype and spleen weight for Experiment 1 (left) and Experiment 2 (right). Cummulative score for erythema, scaling, and thickness over time (top), and ratio of spleen to body weight ratio (bottom).

FIG. 5 is a drawing showing cytokine induction and complement activation. The back skin of each mouse was removed on day 10 (Experiment 1) or day 6 (Experiment 2), and assayed for TNF alpha, IL17, IL23 and C5b-9 terminal complement complex. The level of cytokine was normalized against the weight of the skin. Statistical significance was determined by Student t test.

FIGS. 6A to 6C are drawings showing scaling, erythema, and thickness of the back skin scored independently daily on a scale from 0 to 4.

FIG. 6A is a drawing showing scaling score of the back skin.

FIG. 6B is a drawing showing thickness score of the back skin.

FIG. 6C is a drawing showing erythema score of the back skin.

FIG. 6D is a drawing showing cumulative score of the back skin. The cumulative score (0-12) was the sum of the scores from scaling, erythema, and thickness of the back skin.

FIG. 7 is a drawing showing cytokine induction and complement activation. The back skin of each mouse was removed on day 10, and assayed for TNF alpha, IL17, IL23 and C5b-9 terminal complement complex. The level of cytokine was normalized against the weight of the skin. Two independent experiments were performed using ten mice per group in each of the experiments. Statistical significance was determined by Student t test.

### DETAILED DESCRIPTION

We disclose a topical application of an exosome formulation to treat dermatological immune diseases/disorders such as psoriasis or eczema by alleviating dermal immune molecules or complexes.

This formulation could also be used together with topical application of yolk lecithin liposomes (YLLs) to synergistically alleviate a wider spectrum of dermal immune molecules or complexes.

MSC exosomes are 50-200 nm extracellular vesicles secreted by mesenchymal stem cells. They have immunomodulatory activity^{1,2} and could attenuate inflammation to restore homeostasis. YLL of 100 to 200 nm have been shown to be highly compatible with human skin and could enhance skin barrier³.

We demonstrate that in a mouse model of IMQ-induced psoriasis, topical application of a specific formulation of MSC exosomes alone reduced TNFα and terminal complement complex C5b-9. As topical application of a YLL cream have been previously shown to reduce TNF-α with a moderate effect on reducing IL-23³, we applied exosome and YLL together and observed that this combination enhanced a statistically significant reduction in TNFα, IL-17, IL-23, and Membrane Attack Complex C5b-9. Furthermore, the exosome formulation is stable and could be stored at ambient temperature.

### Exosomes

The exosome for use in the methods and compositions describe here may be derived from a number of different sources. Exosomes from different sources may be suitable for lyophilisation according to the methods disclosed in this document.

The exosome may for example be derivable from a stem cell such as a mesenchymal stem cell (MSC), as described below and in WO 2009/105044.

Exosomes are small membrane vesicles formed in late endocytic compartments (multivesicular bodies) first described to be secreted by reticulocytes in 1983 and subsequently found to be secreted by many cells types including various haematopoietic cells, tumours of haematopoietic or non-haematopoietic origin and epithelial cells. They are distinct entities from the more recently described ‘fibonuclease complex’ also named exosome.

Exosomes may be defined by a number of morphological and biochemical parameters. Accordingly, the exosome described here may comprise one or more of these morphological or biochemical parameters.

Exosomes are classically defined as “saucer-like” vesicles or a flattened sphere limited by a lipid bilayer with diameters of 40-100 nm and are formed by inward budding of the endosomal membrane. Like all lipid vesicles and unlike protein aggregates or nucleosomal fragments that are released by apoptotic cells, exosomes have a density of ˜1.13-1.19 g/ml and float on sucrose gradients. Exosomes are enriched in cholesterol and sphingomyelin, and lipid raft markers such as GM1, GM3, flotillin and the src protein kinase Lyn suggesting that their membranes are enriched in lipid rafts.

The molecular composition of exosomes from different cell types and of different species has been examined. In general, exosomes contain ubiquitous proteins that appear to be common to all exosomes and proteins that are cell-type specific. Also, proteins in exosomes from the same cell-type but of different species are highly conserved. The ubiquitous exosome-associated proteins include cytosolic proteins found in cytoskeleton e.g. tubulin, actin and actin-binding proteins, intracellular membrane fusions and transport e.g. annexins and rab proteins, signal transduction proteins e.g. protein kinases, 14-3-3 and heterotrimeric G proteins, metabolic enzymes e.g. peroxidases, pyruvate and lipid kinases, and enolase-1 and the family of tetraspanins e.g. CD9, CD63, CD81 and CD82. The tetraspannins are highly enriched in exosomes and are known to be involved in the organization of large molecular complexes and membrane subdomains.

Examples of cell-type specific proteins in exosomes are MHC class II molecules in exosomes from MHC class II-expressing cells, CD86 in dendritic cell-derived exosomes, T-cell receptors on T-cell-derived exosomes etc. Notably, exosomes do not contain proteins of nuclear, mitochondrial, endoplasmic-reticulum or Golgi-apparatus origin. Also, highly abundant plasma membrane proteins are absent in exosomes suggesting that they are not simply fragments of the plasma membrane. Many of the reported ubiquitous exosome-associated proteins are also present in the proteomic profile of the hESC-MSC secretion.

Exosomes are also known to contain mRNA and microRNA, which can be delivered to another cell, and can be functional in this new location. The physiological functions of exosome remain poorly defined. It is thought to help eradicate obsolete proteins, recycle proteins, mediate transmission of infectious particles such as prions and viruses, induce complement resistance, facilitate immune cell-cell communication and transmit cell signaling. Exosomes have been used in immunotherapy for treatment of cancer.

Exosome Molecular Weight

The exosome may have a molecular weight of greater than 100 kDa. It may have a molecular weight of greater than 500 kDa. For example, it may have a molecular weight of greater than 1000 kDa.

The molecular weight may be determined by various means. In principle, the molecular weight may be determined by size fractionation and filtration through a membrane with the relevant molecular weight cut-off. The exosome size may then be determined by tracking segregation of component proteins with SDS-PAGE or by a biological assay.

Assay of Molecular Weight by SDS-PAGE

The exosome may have a molecular weight of greater than 100 kDa. For example, the exosome may be such that most proteins of the exosome with less than 100 kDa molecular weight segregate into the greater than 100 kDa molecular weight retentate fraction, when subject to filtration. Similarly, when subjected to filtration with a membrane with a 500 kDa cut off, most proteins of the exosome with less than 500 kDa molecular weight may segregate into the greater than 500 kDa molecular weight retentate fraction. This indicates that the exosome may have a molecular weight of more than 500 kDa.

Assay of Molecular Weight by Biological Activity

The exosome may have a molecular weight of more than 1000 kDa. For example, the exosome may be such that when subject to filtration with a membrane with a molecular weight cutoff of 1000 kDa, the relevant biological activity substantially or predominantly remains in the retentate fraction. Alternatively or in addition, biological activity may be absent in the filtrate fraction. The biological activity may comprise any of the biological activities of the exosome described elsewhere in this document.

Assay of Molecular Weight by Infarct Size

For example, the biological activity may comprise reduction of infarct size, as assayed in any suitable model of myocardial ischemia and reperfusion injury. For example, the biological activity may be assayed in a mouse or pig model, as described in WO 2009/105044.

In summary, myocardial ischemia is induced by 30 minutes left coronary artery (LCA) occlusion by suture ligation and reperfusion is initiated by removal of suture. Mice are treated with liquid containing the exosomes (such as unfractionated MSC-CM), filtrate (such as <100 or 1,000 kD fraction), retentate (such as >1000 kD retentate) or saline intravenously via the tail vein, 5 minutes before reperfusion. 24 hours later, the hearts are excised. Before excision, the Area At Risk (AAR) is determined by religating the LCA and then perfusing Evans blue through the aorta.

AAR is defined as the area not stained by the dye and is expressed as a percentage of the left ventricular wall area. Infarct size is assessed 24 hours later using Evans blue and TTC. Where the relative infarct size is significantly reduced in animals treated with mesenchymal stem cell conditioned medium (MSC-CM) and the retentate (such as a >1000 kD) fraction when compared to saline, this indicates that the exosome has a molecular weight which is higher than the relevant cutoff of the membrane (e.g., greater than 1000 kDa).

Exosome Size

The exosome may have a size of greater than 2 nm. The exosome may have a size of greater than 5 nm, 10 nm, 20 nm, 30 nm, 40 nm or 50 nm. The exosome may have a size of greater than 100 nm, such as greater than 150 nm. The exosome may have a size of substantially 200 nm or greater.

The exosome may have a range of sizes, such as between 2 nm to 20 nm, 2 nm to 50 nm, 2 nm to 100 nm, 2 nm to 150 nm or 2 nm to 200 nm. The exosome may have a size between 20 nm to 50 nm, 20 nm to 100 nm, 20 nm to 150 nm or 20 nm to 200 nm. The exosome may have a size between 50 nm to 100 ηm, 50 ηm to 150 nm or 50 nm to 200 nm. The exosome may have a size between 100 nm to 150 nm or 100 nm to 200 nm. The exosome may have a size between 150 nm to 200 nm.

The size may be determined by various means. In principle, the size may be determined by size fractionation and filtration through a membrane with the relevant size cut-off. The exosome size may then be determined by tracking segregation of component proteins with SDS-PAGE or by a biological assay.

The size may also be determined by electron microscopy.

The size may comprise a hydrodynamic radius. The hydrodynamic radius of the exosome may be below 100 nm. It may be between about 30 nm and about 70 nm. The hydrodynamic radius may be between about 40 nm and about 60 nm, such as between about 45 nm and about 55 nm. The hydrodynamic radius may be about 50 nm.

The hydrodynamic radius of the exosome may be determined by any suitable means, for example, laser diffraction or dynamic light scattering. An example of a dynamic light scattering method to determine hydrodynamic radius is described in WO 2009/105044.

### Obtaining Mesenchymal Stem Cells (MSC)

Mesenchymal stem cell particles such as exosomes may be isolated or produced, using the methods described here, from mesenchymal stem cell conditioned medium (MSC-CM).

MSCs suitable for use in the production of conditioned media and exosomes may be made by any method known in the art.

In particular, MSCs may be made by propagating a cell obtained by dispersing an embryonic stem (ES) cell colony, or a descendent thereof, in the absence of co-culture in a serum free medium comprising FGF2. This is described in detail in the sections below.

Methods of obtaining mesenchymal stem cells (MSC) or MSC-like cells from hESCs may involve either transfection of a human telomerase reverse transcriptase (hTERT) gene into differentiating hESCs (Xu et al., 2004) or coculture with mouse OP9 cell line (Barberi et al., 2005). The use of exogenous genetic material and mouse cells in these derivation protocols introduces unacceptable risks of tumorigenicity or infection of xenozootic infectious agents.

The exosomes may therefore be made from MSCs derived by the use of a clinically relevant and reproducible protocol for isolating similar or identical (such as homogenous) MSC populations from differentiating hESCs. In general, the method comprises dispersing an embryonic stem (ES) cell colony into cells. The cells are then plated out and propagated. The cells are propagated in the absence of co-culture in a serum free medium comprising fibroblast growth factor 2 (FGF2), in order to obtain mesenchymal stem cells (MSCs).

Thus, the protocol does not require serum, use of mouse cells or genetic manipulations and requires less manipulations and time, and is therefore highly scalable. The protocol may be used for the isolation of MSCs from two different hESC lines, HuES9 and H-1 and also a third one, Hes-3. Human ES cell derived MSCs (hESC-MSCs) obtained by the methods and compositions described here are remarkably similar to bone-marrow derived MSCs (BM-MSCs).

The embryonic stem cell culture may comprise a human embryonic stem cell (hESC) culture.

In a one embodiment, a method of generating mesenchymal stem cells (MSC) comprises trypsinizing and propagating hESCs without feeder support in media supplemented with FGF2 and optionally PDGF AB before sorting for CD105+CD34− cells.

The method may comprise sorting for CD105+, CD34− cells from trypsinized hESCs one week after feeder-free propagation in a media supplemented with FGF2 and optionally PDGF AB will generate to generate a hESC-MSC cell culture in which at least some, such as substantially all, or all cells are similar or identical (such as homogenous) to each other.

The MSCs produced by this method may be used to produce mesenchymal stem cell conditioned medium (MSC-CM), from which the exosomes may be isolated.

### Exosome Preparation

Exosomes may be prepared according to the methods disclosed in International Patent Publication Number WO 2009/105044.

Ion exchange chromatography, as described in International Patent Publication WO 2012/087241, may also be used to separate and/or purify exosomes from mesenchymal stem cells.

Example Protocol (Ion Exchange Chromatography)

The following paragraphs provide a specific example of how a mesenchymal stem cell exosome may be obtained using ion exchange chromatography.

A mesenchymal stem cell exosome may be produced by culturing mesenchymal stem cells in a medium to condition it. The mesenchymal stem cells may comprise HuES9.E1 cells. The medium may comprise DMEM. The DMEM may be such that it does not comprise phenol red. The medium may be supplemented with insulin, transferrin, or selenoprotein (ITS), or any combination thereof. It may comprise FGF2. It may comprise PDGF AB. The concentration of FGF2 may be about 5 ng/ml FGF2. The concentration of PDGF AB may be about 5 ng/ml. The medium may comprise glutamine-penicillin-streptomycin or -mercaptoethanol, or any combination thereof.

The cells may be cultured for about 1, 2, 3, 4, 5, 6, 7, 8, 9, 10 days or more, for example 3 days. The conditioned medium may be obtained by separating the cells from the medium. The conditioned medium may be centrifuged, for example at 500 g. it may be concentrated by filtration through a membrane. The membrane may comprise a >1000 kDa membrane. The conditioned medium may be concentrated about 50 times or more.

The conditioned medium may then be subjected to anion exchange chromatography, as described in the Examples.

In addition to detecting the alpha subunit of the 20S proteasome and CD9, UV absorbance such as at 220 nm may also be used to track the progress of elution. Fractions may be examined for dynamic light scattering (DLS) using a quasi-elastic light scattering (QELS) detector.

Fractions which are found to exhibit dynamic light scattering may be retained. In addition to anion exchange chromatography, the conditioned medium may be separated by size exclusion, as an additional step. Any size exclusion matrix such as Sepharose may be used. As an example, a TSK Guard column SWXL, 6×40 mm or a TSK gel G4000 SWXL, 7.8×300 mm may be employed. The eluent buffer may comprise any physiological medium such as saline. It may comprise 20 mM phosphate buffer with 150 mM of NaCl at pH 7.2. The chromatography system may be equilibrated at a flow rate of 0.5 ml/min. The elution mode may be isocratic.

For example, a fraction which is produced by the general method as described above, and which elutes with a retention time of 11-13 minutes, such as 12 minutes, is found to exhibit dynamic light scattering. The rh of exosomes in this peak is about 45-55 nm. Such fractions comprise mesenchymal stem cell exosomes.

### Lyophilisation of Exosomes

In order for the pharmaceutical compositions described here to be prepared, exosomes may be prepared fresh or they may be obtained from long term storage, such as in the form of lyophilised specimens.

Methods for the lyophilisation of exosomes are known in the art, and are described in detail for example in International Patent Application Number PCT/SG2017/050513, published as WO 2018/070939.

An example protocol for lyophilisation of an exosome may comprise providing an exosome suspension in a lyophilisation buffer comprising a sugar at less than 10% w/v, freezing the exosome suspension and removing water from the frozen suspension by freeze drying.

The lyophilisation buffer may comprise a sugar at a concentration of 9% or less. It may include other compounds such as sugars or polyols, polymers, surfactants, buffers, amino acids, chelating complexes and inorganic salts. Compounds such as acetone or poloxamer 188 may also be included. The compounds may perform a function as an excipient, for example.

The lyophilisation buffer may include for example a combination of lactose and glucose at a specified mass ratio such as 2:1 glucose:lactose. The sugar may comprise a mono-saccharide or a di-saccharide. Examples of sugars suitable for use include trehalose, sucrose, lactose, glucose, saccharose, maltose and mannitol. The sugar may comprise a di-saccharide such as trehalose, sucrose or sorbitol at a concentration of 4% w/v.

The lyophilisation may be such that the lyophilised exosome exhibits at least one biological activity of an exosome which may comprise a biochemical activity such as CD73 enzymatic activity. The biological activity may comprise a therapeutic activity, such as cardioprotective activity and the exosome may exhibit 5% or more of the biological activity when reconstituted.

The exosome may exhibit the biological activity when stored at a temperature of 0° C. or higher. The exosome may exhibit the biological activity when stored for 1 day or more. The exosome may exhibit 20% or more CD73 enzymatic activity. The exosome may exhibit 20% or more activity to induce polarization of CD4+ T cells to regulatory T cells (Tregs). The biological activity may comprise cardioprotection. The cardioprotection may be assayed using an ex vivo Landerdorf model of ischemia-reperfusion injury. The cardioprotection may be assayed by the degree of hypercontracture over post-reperfusion time. The exosome may exhibit 20% or more cardioprotective activity.

The freezing step may comprise freezing the exosome suspension at a temperature of −10° C. or lower for 1 hour or more. The removal of water step may comprise freeze drying at a temperature of −10° C. or lower such as at a pressure of 1 mbar or lower. The removal of water step may comprise freeze drying at a temperature of −10° C. or lower, for example for 5 hours or longer.

The freezing step may comprise freezing the exosome suspension at about −20° C. for about 3 hours. The removal of water step may comprise freeze drying at <0.05 mbar for about 12 to 24 hours. The method may further comprise a step before the removal of water step of transferring the frozen suspension to a freeze dryer pre-cooled to −48° C.

The lyophilised exosome may be stored at a temperature of 4° C. or higher. The lyophilised exosome may be stored in substantially dry conditions, such as in a desiccator, for example in air with a relative humidity of 5% or less or in an inert gas such as nitrogen.

The lyophilisation buffer may further comprise a phosphate buffer which may be a pH 6.5-7.5 buffer. The lyophilisation buffer may further comprise an amino acid which may comprise L-glutamic acid such as at a concentration of 0.0049M. The lyophilisation buffer may further comprise a protein binding stabiliser which may comprise human serum albumin such as at a concentration of 1% w/v.

The method may further comprise a step of viral clearance which may be by exposure to gamma radiation, X-ray irradiation or ethylene oxide.

### Storage of Exosomes in Oil

Exosomes may be conveniently stored as a suspension or dispersion in oil.

We describe a method of storage of an exosome, for example a mesenchymal stem cell (MSC) exosome in the form of a suspension or dispersion of the exosome in an oil.

For this purpose, freshly prepared exosomes or lyophilised exosomes may be suspended or dispersed in oil. The oil may comprise any suitable oil, such as a plant oil.

Examples of plant oils include almond oil, amaranth oil, amur cork tree fruit oil, apple seed oil, apricot oil, argan oil, artichoke oil, Astrocaryum murumuru butter, Astrocaryum vulgare (tucumã) oil, avocado oil, açai oil, babassu oil, balanos oil, beech nut oil, ben oil, bitter gourd oil, black seed oil, blackcurrant seed oil, bladderpod oil, borage seed oil, Borneo tallow nut oil, bottle gourd oil, Brazil nut oil, Brucea javanica oil, buffalo gourd oil, burdock oil (bur oil), buriti oil, butternut squash seed oil, candlenut oil (kukui nut oil), canola oil, cape chestnut oil, carob pod oil, carrot seed oil, cashew oil, castor oil, chaulmoogra oil, citrus oil, cocklebur oil, coconut oil (copra oil), cohune oil, colza oil, copaiba, coriander seed oil, corn oil, cottonseed oil, crambe oil, croton oil (Tiglium oil), cuphea oil, cupuaçu butter, dammar oil, date seed oil, dika oil, egusi seed oil, evening primrose oil, false flax oil, flaxseed oil, grape seed oil, grapefruit seed oil, hazelnut oil, hemp oil, honesty oil, illipe butter, jatropha oil, jojoba oil, kapok seed oil, kenaf seed oil, lallemantia oil, lemon oil, linseed oil, macadamia oil, mafura oil, mango oil, manila oil, meadowfoam seed oil, milk bush oil, mongongo nut oil, mowrah butter, mustard oil, nahor oil, neem oil, niger seed oil, nut oil, nutmeg butter, nutmeg oil, of on oil, okra seed oil, olive oil, orange oil, palm oil, papaya seed oil, paradise oil, passion fruit oil, peanut oil (ground nut oil), pecan oil, pequi oil, perilla seed oil, persimmon seed oil, petroleum nut oil, pili nut oil, pine nut oil, pistachio oil, pomegranate seed oil, pongamia oil (honge oil), poppyseed oil, pracaxi oil, prune kernel oil, pumpkin seed oil, quinoa oil, radish oil, ramtil oil, rapeseed oil, rice bran oil, rose hip seed oil, royle oil, rubber seed oil, sacha inchi oil, safflower oil, Salicornia oil, sapote oil, sea buckthorn oil, sea rocket seed oil, seje oil, sesame oil, shea butter, snowball seed oil (Viburnum oil), soybean oil, Stillingia oil (Chinese vegetable tallow oil), sunflower oil, tall oil, tamanu or foraha oil, taramira oil, tea seed oil (Camellia oil), thistle oil, tigernut oil (nut-sedge oil), tobacco seed oil, tomato seed oil, tonka bean oil (cumaru oil), tung oil, ucuhuba seed oil, vernonia oil, virgin pracaxi oil, walnut oil, watermelon seed oil, wheat germ oil and yangu oil.

The exosomes may be suspended or dispersed in for example olive oil, palm oil, soy oil or coconut oil. The exosomes may be suspended or dispersed in the oil in any suitable proportion.

The exosome suspension or dispersion in oil may be stored, for example at room temperature, prior to use.

The exosome suspension or dispersion in oil may be such that the exosome exhibits at least one biological activity of an exosome following a period of storage. The biological activity may comprise a biochemical activity such as CD73 enzymatic activity. The biological activity may comprise a therapeutic activity, such as cardioprotective activity.

The exosome may exhibit 5% or more of the biological activity following storage. The exosome may exhibit 10% or more of the biological activity following storage. The exosome may exhibit 15% or more of the biological activity following storage. The exosome may exhibit 20% or more of the biological activity following storage. The exosome may exhibit 25% or more of the biological activity following storage. The exosome may exhibit 30% or more of the biological activity following storage. The exosome may exhibit 35% or more of the biological activity following storage. The exosome may exhibit 40% or more of the biological activity following storage. The exosome may exhibit 45% or more of the biological activity following storage. The exosome may exhibit 50% or more of the biological activity following storage. The exosome may exhibit 55% or more of the biological activity following storage. The exosome may exhibit 60% or more of the biological activity following storage.

The exosome may exhibit 65% or more of the biological activity following storage. The exosome may exhibit 70% or more of the biological activity following storage. The exosome may exhibit 75% or more of the biological activity following storage. The exosome may exhibit 80% or more of the biological activity following storage. The exosome may exhibit 85% or more of the biological activity following storage. The exosome may exhibit 90% or more of the biological activity following storage. The exosome may exhibit or 95% or more of the biological activity following storage.

The exosome may exhibit the biological activity when stored at a temperature of 0° C. or higher. The exosome may exhibit the biological activity when stored at a temperature of 4° C. or higher. The exosome may exhibit the biological activity when stored at a temperature of 10° C. or higher. The exosome may exhibit the biological activity when stored at a temperature of 15° C. or higher. The exosome may exhibit the biological activity when stored at a temperature of 20° C. or higher. The exosome may exhibit the biological activity when stored at a temperature of 25° C. or higher. The exosome may exhibit the biological activity when stored at a temperature of 30° C. or higher. The exosome may exhibit the biological activity when stored at a temperature of 35° C. or higher. The exosome may exhibit the biological activity when stored at a temperature of 40° C. or higher.

The exosome may exhibit the biological activity when stored for 1 day or more. The exosome may exhibit the biological activity when stored for 5 days or more. The exosome may exhibit the biological activity when stored for 7 days or more. The exosome may exhibit the biological activity when stored for 10 days or more. The exosome may exhibit the biological activity when stored for 14 days or more. The exosome may exhibit the biological activity when stored for 20 days or more. The exosome may exhibit the biological activity when stored for 21 days or more. The exosome may exhibit the biological activity when stored for 25 days or more. The exosome may exhibit the biological activity when stored for 28 days or more. The exosome may exhibit the biological activity when stored for 30 days or more. The exosome may exhibit the biological activity when stored for 35 days or more. The exosome may exhibit the biological activity when stored for or 40 days or more.

The biological activity may comprise CD73 enzymatic activity.

The exosome may exhibit 20% or more CD73 enzymatic activity. The exosome may exhibit 25% or more CD73 enzymatic activity. The exosome may exhibit 30% or more CD73 enzymatic activity. The exosome may exhibit 35% or more CD73 enzymatic activity. The exosome may exhibit 40% or more CD73 enzymatic activity. The exosome may exhibit 45% or more CD73 enzymatic activity. The exosome may exhibit 50% or more CD73 enzymatic activity. The exosome may exhibit 55% or more CD73 enzymatic activity. The exosome may exhibit 60% or more CD73 enzymatic activity. The exosome may exhibit that CD73 enzymatic activity after 55 days of storage at 40° C. The exosome may exhibit that CD73 enzymatic activity as compared to the activity after 1 day of storage at 40° C.

The biological activity may comprise induction of polarization of CD4+ T cells to regulatory T cells (Tregs).

The exosome may exhibit 20% or more activity to induce polarization of CD4+ T cells to regulatory T cells (Tregs). The exosome may exhibit 25% or more activity to induce polarization of CD4+ T cells to regulatory T cells (Tregs). The exosome may exhibit 30% or more activity to induce polarization of CD4+ T cells to regulatory T cells (Tregs). The exosome may exhibit 35% or more activity to induce polarization of CD4+ T cells to regulatory T cells (Tregs). The exosome may exhibit 40% or more activity to induce polarization of CD4+ T cells to regulatory T cells (Tregs). The exosome may exhibit 45% or more activity to induce polarization of CD4+ T cells to regulatory T cells (Tregs). The exosome may exhibit 50% or more activity to induce polarization of CD4+ T cells to regulatory T cells (Tregs). The exosome may exhibit 55% or more activity to induce polarization of CD4+ T cells to regulatory T cells (Tregs). The exosome may exhibit 60% or more activity to induce polarization of CD4+ T cells to regulatory T cells (Tregs). The exosome may exhibit that activity to induce polarization of CD4+ T cells to regulatory T cells (Tregs) after 3 weeks of storage at 40° C. The exosome may exhibit that activity to induce polarization of CD4+ T cells to regulatory T cells (Tregs) when compared with a non-lyophilized exosome stored at −80° C.

The biological activity may comprise cardioprotection. The cardioprotection may be assayed using an ex vivo Landerdorf model of ischemia-reperfusion injury. The cardioprotection may be assayed by the degree of hypercontracture over post-reperfusion time.

The exosome may exhibit 20% or more cardioprotective activity. The exosome may exhibit 25% or more cardioprotective activity. The exosome may exhibit 30% or more cardioprotective activity. The exosome may exhibit 35% or more cardioprotective activity. The exosome may exhibit 40% or more cardioprotective activity. The exosome may exhibit 45% or more cardioprotective activity. The exosome may exhibit 50% or more cardioprotective activity. The exosome may exhibit 55% or more cardioprotective activity. The exosome may exhibit 60% or more cardioprotective activity. The exosome may exhibit that cardioprotective activity when compared with exosome stored at −80° C.

### Exosome Emulsions

The pharmaceutical compositions described here comprise emulsions containing exosomes. Such emulsions may comprise oil-in-water emulsions of exosomes.

The emulsions may be made by means known in the art. They may be made from freshly prepared exosomes, or lyophilised exosomes or exosomes stored in oil.

Emulsions are heterogeneous systems composed of at least two immiscible liquids, for example water and oil, one of which is usually uniformly dispersed as fine droplets throughout the other liquid phase by a mechanical agitation process.

The phase existing as small droplets is called the dispersed phase and the surrounding liquid is known as the continuous phase. The dispersed phase (internal phase) is therefore composed of small globules of a liquid distributed throughout a liquid vehicle (external or continuous phase) in which it is immiscible.

Emulsions having an oleagenous internal phase and an aqueous external phase are designated oil-in-water (o/w) emulsions. On the other hand, water-in-oil (w/o) emulsions have an aqueous internal phase and an oleaginous external phase.

In order to prepare a stable emulsion an emulsifying agent as well as energy in the form of work is required.

Emulsions are thermodynamically unstable as the dispersed and continuous phases can revert back as separate phases, oil and water, by fusion or coalescence of droplets. However, emulsions are commonly stabilized by an emulsifying agent, often referred to as a surfactant.

In general, after vigorous agitation of the two immiscible phases, the more rapidly coalescing droplets form the continuous phase. This is usually the liquid that is present in the larger amount—the greater the number of droplets, the higher the probability of collision and coalescence.

Methods of emulsification and production of oil-in-water emulsions are known in the art. Such methods include the use of high-pressure homogenizers, ultrasound homogenizers, colloid mills, ball and roller mills, rotor/stator systems such as stirred vessels and electrical and condensation devices.

Exosome emulsions may be prepared by homogenising exosomes in a suitable emulsifier.

### Emulsifier

An emulsifier (also known as an “emulgent”) is a substance that stabilizes an emulsion by increasing its kinetic stability.

The emulsifier may comprise a natural emulsifying agent, a semi-synthetic emulsifying agent or a synthetic emulsifying agent.

Natural emulsifying agents include agar, tragacanth, alginates, gum, pectins, acacia, starch, gelatin, albumin, lecithin and cholesterol.

Semi-synthetic emulsifying agents include methylcellulose derivatives such as carboxymethylcellulose and starch derivatives such as octenyl succinate anhydride treated starch, also known as OSA-starch.

Synthetic emulsifying agents include benzalkonium chloride, benzethonium chloride, sodium oleate potassium oleate, triethanolamine stearate, detergents such as sodium lauryl sulfate, sodium dioctyl sulfosuccinate, sodium docusate), sorbitan esters (Spans) and polyoxyethylene derivatives of sorbitan esters (Tweens) and glyceryl esters.

### Reduction of Inflammatory Cytokine Levels

The exosome emulsion described here may be capable of reducing the level of an inflammatory cytokine when applied to a patient.

Thus, the application of the pharmaceutical composition to an individual may be such as to result in reduced level of an inflammatory cytokine in the individual, as compared to an individual to which the pharmaceutical composition is not applied.

The method may be such that the application of the pharmaceutical composition alleviates immune reactivity triggered by exposure of the skin of an individual to an irritant. The irritant may comprise Imiquimod (IMQ).

The individual may be suffering from a skin-related disease. The skin-related disease may comprise dermatitis or psoriasis. Administration of the pharmaceutical composition may reduce or alleviate erythema. It may reduce or alleviate scaling. It may reduce or alleviate thickness in the individual. It may do one or more of the above.

The inflammatory cytokine may comprise any suitable cytokine, for example TNFα, IL-17, IL-23 and Membrane Attack Complex C5b-9. The reduction in inflammatory cytokine level may be as compared to an individual to which the pharmaceutical composition is not applied.

Means for measuring levels of inflammatory cytokine levels are known in the art and may be applied as needed.

The exosome emulsion may be capable of reducing the level of an inflammatory cytokine by 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more or 100% or more, as compared to an individual to which the pharmaceutical composition is not applied.

### Lecithin Containing Liposomes

The pharmaceutical composition as set out in detail in this document may further comprise a liposome comprising a lecithin.

We describe a combination of a pharmaceutical composition as set out in detail in this document comprising an oil-in-water emulsion of an exosome, together with a liposome comprising a lecithin. The lecithin may comprise egg yolk lecithin. The liposome may comprise a yolk lecithin liposome (YLL).

Lecithin containing liposomes are described in detail in International Patent Application PCT/SG2019/050081, published as WO 2019/160502.

The lecithin may comprise egg yolk lecithin. The liposome may comprise a yolk lecithin liposome (YLL).

The liposomes may have a medial size of about 100 nm, such as 105.5 nm. The liposome may comprise about 0.13% w/v protein, about 0.02% w/v carbohydrates and about 1.03% w/v fats. The liposome may comprise about 7.62 mM phospholipids, about 134.82 sphingomyelin, about 1.12 mM triglycerides and about 5.48 mM cholesterol.

The liposome may comprise choline (about 0.132 mg/ml), Vitamin A (about 0.819 iu/ml), Vitamin D3 (cholecalciferol, about 0.513 iu/ml) and Vitamin E (a-tocopherol acetate, about 0.126 mg/ml). The liposome may comprise phosphatidylcholine (about 56%), free fatty acids (about 11.9%), phosphatidylethanolamine (about 9.6%), cholesterol (about 9.3%), triglycerides (about 5.9%), sphingomyelin (about 2.5%) and lysophosphatidycholine (about 2.3%). The liposome may comprise oleic acid (cis18:1), palmitic acid (16:0), linoleic acid (18:2) and stearic acid (18:0).

Liposome synthesis is a well-established procedure and protocols for making liposome are universally available (Akbarzadeh A, Rezaei-Sadabady R, Davaran S, Joo S W, Zarghami N, Hanifehpour Y, et al. Liposome: classification, preparation, and applications. Nanoscale Research Letters. 2013; 8(1): 102.).

The synthesis of liposomes from egg yolk lecithin is described for example in U.S. Pat. No. 5,429,823). A group of Japanese high school children have reported synthesizing liposomes using egg yolk lecithin (Sugiyama and Sadzuka, 2011. Preparation of liposome using egg yolk. Yakugaku Zasshi. 2011; 131(10):1519-25).

Yolk lecithin liposomes may be made using any of the established methods as described in Akbarzadeh et al. These methods consist of four basic steps: Drying down or solubilizing lipids; dispersing the dried or solubilized lipids in aqueous medium, purifying and sizing the liposome and analyzing the product. A detailed protocol is also readily available on the internet at http://www.avantilipids.com/index.php?option=com_content&view=article&id=1384&Itemid=372.

Alternatively, egg yolk lecithin liposomes are commercially available from commercial sources. For example, egg yolk lecithin liposomes is commercially available as catalogue #ESN13-01 from Vesiderm Pte Ltd (Singapore).

### Dermatological Conditions

We disclose the use of an emulsion of an exosome, such as a mesenchymal stem cell (MSC) exosome, for the treatment or prevention of a dermatological disease or condition. The dermatological disease or condition may comprise a dermatological immune disease.

Examples of dermatological diseases and conditions are known in the art and include for example alopecia areata, atopic dermatitis, bullous pemphigoid, bullous systemic lupus erythematosus, dermatitis herpetiformis (DH), dermatomyositis, drug-induced pemphigus, eczema, epidermolysis bullosa acquisita (EBA), IgA pemphigus, lichen sclerosus, linear IgA bullous dermatosis, mucous membrane pemphigoid, paraneoplastic pemphigus, pemphigoid gestationis, pemphigus, pemphigus erythematosus (PE), pemphigus foliaceus, pemphigus vegetans, pemphigus vulgaris, psoriasis, scleroderma, systemic sclerosis and vitiligo.

### Eczema

Atopic dermatitis (eczema) is a condition that makes skin red and itchy. Atopic dermatitis is common in children but can occur at any age. Atopic dermatitis is long lasting (chronic) and tends to flare periodically. It may be accompanied by asthma or hay fever.

Atopic dermatitis (eczema) signs and symptoms vary widely from person to person and include: dry skin, itching, which may be severe, especially at night, red to brownish-gray patches, especially on the hands, feet, ankles, wrists, neck, upper chest, eyelids, inside the bend of the elbows and knees, and in infants, the face and scalp, small, raised bumps, which may leak fluid and crust over when scratched, thickened, cracked, scaly skin and raw, sensitive, swollen skin from scratching.

Atopic dermatitis most often begins before age 5 and may persist into adolescence and adulthood. For some people, it flares periodically and then clears up for a time, even for several years.

### Psoriasis

The compositions and methods of our invention may be used for the treatment or alleviation of symptoms of psoriasis.

Psoriasis manifests itself as inflamed swollen skin lesions covered with silvery white scale. Characteristics of psoriasis include pus-like blisters (pustular psoriasis), severe sloughing of the skin (erythrodermic psoriasis), drop-like dots (guttate psoriasis) and smooth inflamed lesions (inverse psoriasis).

The causes of psoriasis are currently unknown, although it has been established as an autoimmune skin disorder with a genetic component. One in three people report a family history of psoriasis, but there is no pattern of inheritance. However, there are many cases in which children with no apparent family history of the disease will develop psoriasis. Whether a person actually develops psoriasis may depend on “trigger factors” which include systemic infections such as strep throat, injury to the skin (the Koebner phenomenon), vaccinations, certain medications, and intramuscular injections or oral steroid medications. Once something triggers a person's genetic tendency to develop psoriasis, it is thought that in turn, the immune system triggers the excessive skin cell reproduction.

Skin cells are programmed to follow two possible programs: normal growth or wound healing. In a normal growth pattern, skin cells are created in the basal cell layer, and then move up through the epidermis to the stratum corneum, the outermost layer of the skin. This normal process takes about 28 days from cell birth to death. When skin is wounded, a wound healing program (regenerative maturation) is triggered, in which cells are produced at a much faster rate, the blood supply increases and localized inflammation occurs. Lesional psoriasis is characterized by cell growth in the alternate growth program. Skin cells (keratinocytes) switch from the normal growth program to regenerative maturation, cells are created and pushed to the surface in as little as 2-4 days, and the skin cannot shed the cells fast enough. The excessive skin cells build up and form elevated, scaly lesions. The white scale (“plaque”) that usually covers the lesion is composed of dead skin cells, and the redness of the lesion is caused by increased blood supply to the area of rapidly dividing skin cells.

Psoriasis is a genetically determined disease of the skin characterized by two biological hallmarks. First, there is a profound epidermal hyperproliferation related to accelerated and incomplete differentiation. Second, there is a marked inflammation of both epidermis and dermis with an increased recruitment of T lymphocytes, and in some cases, formation of neutrophil micro abscesses. Many pathologic features of psoriasis can be attributed to alterations in the growth and maturation of epidermal keratinocytes, with increased proliferation of epidermal cells, occurring within 0.2 mm of the skin's surface. Traditional investigations into the pathogenesis of psoriasis have focused on the increased proliferation and hyperplasia of the epidermis. In normal skin, the time for a cell to move from the basal layer through the granular layer is 4 to 5 weeks. In psoriatic lesions, the time is decreased sevenfold to tenfold because of a shortened cell cycle time, an increase in the absolute number of cells capable of proliferating, and an increased proportion of cells that are actually dividing. The hyperproliferative phenomenon is also expressed, although to a substantially smaller degree, in the clinically uninvolved skin of psoriatic patients.

A common form of psoriasis, psoriasis vulgaris, is characterized by well-demarcated erythematous plaques covered by thick, silvery scales. A characteristic finding is the isomorphic response (Koebner phenomenon), in which new psoriatic lesions arise at sites of cutaneous trauma.

Lesions are often localized to the extensor surfaces of the extremities, and the nails and scalp are also commonly involved. Much less common forms include guttate psoriasis, a form of the disease that often erupts following streptococcal pharyngitis, and pustular psoriasis, which is characterized by numerous sterile pustules, often 2 to 5 mm in diameter, on the palms and soles or distributed over the body.

### Pharmaceutical Compositions

We disclose pharmaceutical compositions comprising an oil-in-water emulsion of an exosome, such as a mesenchymal stem cell (MSC) exosome, optionally together with a lecithin liposome such as a yolk lecithin liposome.

While it is possible for the composition comprising the oil-in-water emulsion of an exosome, optionally together with a lecithin liposome such as yolk lecithin liposome to be administered alone, it is preferable to formulate the active ingredient as a pharmaceutical formulation.

The pharmaceutical formulations disclosed here comprise an effective amount of oil-in-water emulsion of an exosome, optionally together with a lecithin liposomes such as yolk lecithin liposomes together with one or more pharmaceutically-acceptable carriers.

An “effective amount” of oil-in-water emulsion of an exosome, optionally together with a lecithin liposomes such as yolk lecithin liposomes is the amount sufficient to restore, maintain or enhance skin barrier function in an individual.

The effective amount will vary depending upon the particular disease or syndrome to be treated or alleviated, as well as other factors including the age and weight of the patient, how advanced the disease etc state is, the general health of the patient, the severity of the symptoms, and whether the oil-in-water emulsion of an exosome, optionally together with a lecithin liposome such as yolk lecithin liposome is being administered alone or in combination with other therapies.

Suitable pharmaceutically acceptable carriers are well known in the art and vary with the desired form and mode of administration of the pharmaceutical formulation. For example, they may include diluents or excipients such as fillers, binders, wetting agents, disintegrators, surface-active agents, lubricants and the like. Typically, the carrier is a solid, a liquid or a vaporizable carrier, or a combination thereof. Each carrier should be “acceptable” in the sense of being compatible with the other ingredients in the formulation and not injurious to the patient. The carrier should be biologically acceptable without eliciting an adverse reaction (e.g. immune response) when administered to the host.

The pharmaceutical compositions include topical formulations which are preferred where the tissue affected is primarily the skin or epidermis (for example, psoriasis and other epidermal diseases such as dermatitis, etc). The topical formulations include those pharmaceutical forms in which the composition is applied externally by direct contact with the skin surface to be treated. A conventional pharmaceutical form for topical application includes a soak, an ointment, a cream, a lotion, a paste, a gel, a stick, a spray, an aerosol, a bath oil, a solution and the like. Topical therapy is delivered by various vehicles, the choice of vehicle can be important and generally is related to whether an acute or chronic disease is to be treated.

Lotions (powder in water suspension) and solutions (medications dissolved in a solvent) are ideal for hairy and intertriginous areas. Ointments or water-in-oil emulsions, are the most effective hydrating agents, appropriate for dry scaly eruptions, but are greasy and depending upon the site of the lesion sometimes undesirable.

As appropriate, they can be applied in combination with a bandage, particularly when it is desirable to increase penetration of the oil-in-water emulsion of an exosome, optionally together with a lecithin liposome such as yolk lecithin liposome composition into a lesion. Creams or oil-in-water emulsions and gels are absorbable and are the most cosmetically acceptable to the patient. (Guzzo et al, in Goodman & Gilman's Pharmacological Basis of Therapeutics, 9th Ed., p. 1593-15950 (1996)). Cream formulations generally include components such as petroleum, lanolin, polyethylene glycols, mineral oil, glycerin, isopropyl palmitate, glyceryl stearate, cetearyl alcohol, tocopheryl acetate, isopropyl myristate, lanolin alcohol, simethicone, carbomen, methylchlorisothiazolinone, methylisothiazolinone, cyclomethicone and hydroxypropyl methylcellulose, as well as mixtures thereof.

Other formulations for topical application include shampoos, soaps, shake lotions, and the like, particularly those formulated to leave a residue on the underlying skin, such as the scalp (Arndt et al, in Dermatology In General Medicine 2:2838 (1993)).

In general, the concentration of the oil-in-water emulsion of an exosome, optionally together with a lecithin liposome such as yolk lecithin liposome composition in the topical formulation is in an amount of about 0.5 to 50% by weight of the composition, preferably about 1 to 30%, more preferably about 2-20%, and most preferably about 5-10%. The concentration used can be in the upper portion of the range initially, as treatment continues, the concentration can be lowered or the application of the formulation may be less frequent. Topical applications are often applied twice daily. However, once-daily application of a larger dose or more frequent applications of a smaller dose may be effective. The stratum corneum may act as a reservoir and allow gradual penetration of a drug into the viable skin layers over a prolonged period of time.

In a topical application, a sufficient amount of the oil-in-water emulsion of an exosome, optionally together with a lecithin liposome such as yolk lecithin liposome must penetrate a patient's skin in order to obtain a desired pharmacological effect. It is generally understood that the absorption of drug into the skin is a function of the nature of the drug, the behaviour of the vehicle, and the skin. Three major variables account for differences in the rate of absorption or flux of different topical drugs or the same drug in different vehicles; the concentration of drug in the vehicle, the partition coefficient of drug between the stratum corneum and the vehicle and the diffusion coefficient of drug in the stratum corneum. To be effective for treatment, a drug must cross the stratum corneum which is responsible for the barrier function of the skin. In general, a topical formulation which exerts a high in vitro skin penetration is effective in vivo. Ostrenga et al (J. Pharm. Sci., 60:1175-1179 (1971) demonstrated that in vivo efficacy of topically applied steroids was proportional to the steroid penetration rate into dermatomed human skin in vitro.

A skin penetration enhancer which is dermatologically acceptable and compatible with the oil-in-water emulsion of an exosome, optionally together with a lecithin liposome such as yolk lecithin liposome can be incorporated into the formulation to increase the penetration of the active compound(s) from the skin surface into epidermal keratinocytes. A skin enhancer which increases the absorption of the active compound(s) into the skin reduces the amount of the oil-in-water emulsion of an exosome, optionally together with a lecithin liposome such as yolk lecithin liposome needed for an effective treatment and provides for a longer lasting effect of the formulation. Skin penetration enhancers are well known in the art. For example, dimethyl sulfoxide (U.S. Pat. No. 3,711,602); oleic acid, 1,2-butanediol surfactant (Cooper, J. Pharm. Sci., 73:1153-1156 (1984)); a combination of ethanol and oleic acid or oleyl alcohol (EP 267,617), 2-ethyl-1,3-hexanediol (WO 87/03490); decyl methyl sulphoxide and Azone® (Hadgraft, Eur. J. Drug. Metab. Pharmacokinet, 21:165-173 (1996)); alcohols, sulphoxides, fatty acids, esters, Azone®, pyrrolidones, urea and polyoles (Kalbitz et al, Pharmazie, 51:619-637 (1996));

Terpenes such as 1,8-cineole, menthone, limonene and nerolidol (Yamane, J. Pharmacy & Pharmocology, 47:978-989 (1995)); Azone® and Transcutol (Harrison et al, Pharmaceutical Res. 13:542-546 (1996)); and oleic acid, polyethylene glycol and propylene glycol (Singh et al, Pharmazie, 51:741-744 (1996)) are known to improve skin penetration of an active ingredient.

Levels of penetration of the oil-in-water emulsion of an exosome, optionally together with a lecithin liposome such as yolk lecithin liposome composition can be determined by techniques known to those of skill in the art. For example, radiolabelling of the active compound, followed by measurement of the amount of radiolabelled compound absorbed by the skin enables one of skill in the art to determine levels of the composition absorbed using any of several methods of determining skin penetration of the test compound. Publications relating to skin penetration studies include Reinfenrath, W G and G S Hawkins. The Weanling Yorkshire Pig as an Animal Model for Measuring Percutaneous Penetration. In: Swine in Biomedical Research (M. E. Tumbleson, Ed.) Plenum, N.Y., 1986, and Hawkins, G. S. Methodology for the Execution of In Vitro Skin Penetration Determinations. In: Methods for Skin Absorption, B W Kemppainen and W G Reifenrath, Eds., CRC Press, Boca Raton, 1990, pp. 67-80; and W. G. Reifenrath, Cosmetics & Toiletries, 110:3-9 (1995).

For some applications, it is preferable to administer a long acting form of the lecithin oil-in-water emulsion of an exosome, optionally together with a liposome such as yolk lecithin liposome composition using formulations known in the arts, such as polymers. The oil-in-water emulsion of an exosome, optionally together with a lecithin liposome such as yolk lecithin liposome can be incorporated into a dermal patch (Junginger, H. E., in Acta Pharmaceutica Nordica 4:117 (1992); Thacharodi et al, in Biomaterials 16:145-148 (1995); Niedner R., in Hautarzt 39:761-766 (1988)) or a bandage according to methods known in the arts, to increase the efficiency of delivery of the drug to the areas to be treated.

Optionally, the topical formulations described here can have additional excipients for example; preservatives such as methylparaben, benzyl alcohol, sorbic acid or quaternary ammonium compound; stabilizers such as EDTA, antioxidants such as butylated hydroxytoluene or butylated hydroxanisole, and buffers such as citrate and phosphate.

Other therapeutic agents suitable for use herein are any compatible drugs that are effective for the intended purpose, or drugs that are complementary to the retinol formulation. As an example, the treatment with a formulation as set out in this document can be combined with other treatments such as a topical treatment with corticosteroids, calcipotrine, coal tar preparations, a systemic treatment with methotrexate, retinoids, cyclosporin A and photochemotherapy. The combined treatment is especially important for treatment of an acute or a severe skin disease. The formulation utilized in a combination therapy may be administered simultaneously, or sequentially with other treatment, such that a combined effect is achieved.

### EXAMPLES

### Example 1. Limited Penetration of Mesenchymal Stem Cell Exosomes

MSC exosomes are small 50-200 nm extracellular vesicles that secreted by MSCs. They are widely known for its potent immunomodulatory properties¹ and have been reported to alleviate severe immune diseases such as GVHD^{15,2}.

As the skin is generally recognized as a highly potent immune organ¹⁶, exosomes have also been reported to be effective against inflammatory skin disorders such as atopic dermatitis¹⁷. Exosomes targeting dermatological immune diseases are delivered either subcutaneously, intravenously or intraperitoneally to ensure maximum access to immune cells.

There have been no reports that the exosome can exert its immunomodulating activity through topical application. This is consistent with the current understanding that MSC exosome is a bi-lipid membrane vesicle that is similar to a liposome.

We expect that the MSC exosomes will burst in the stratum corneum and will not progress beyond the stratum corneum to exert their effects on living cells below the stratum corneum.

Human skin biopsy samples were treated with 50 ul of PBS or fluorescence-labelled exosome for 2 hours at 37° C. incubator. Thereafter, the skin biopsies were gently washed with PBS and placed in OCT quick-freeze medium compound. Samples were either frozen immediately or after an 8 hour-incubation in OCT medium using liquid nitrogen. 3 um sections were made and then subjected to Haematoxylin and Eosin staining (H&E) analysis and green fluorescence screening.

Images were taken using EVOS microscope for H&E and Carl Zeiss microscope for fluorescence (Dapi and FL 488 detection).

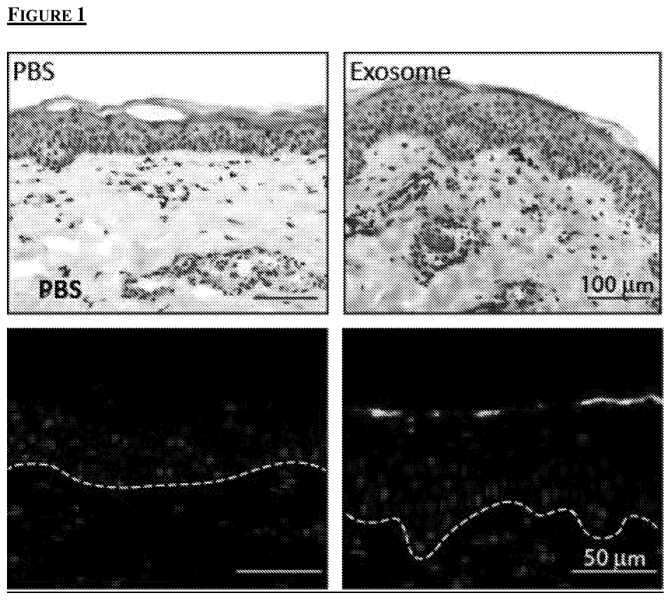

Results are shown in FIG. 1.

Consistent with this, we observed that upon topical application of an aqueous solution of fluorescence-labelled exosome on human skin, the fluorescence only permeated the stratum corneum but did not penetrate cellular layer below the stratum corneum.

### Example 2. Formulation of Oil-in-Water Emulsion of MSC Exosome

We have recently developed an oil-in-water emulsion of MSC exosome which on topical application in a mouse model of Imiquimod (IMQ) Induced Psoriasis reduced disease causing immune molecules or complexes such as TNFalpha and C5b-C9 membrane attack complex.

The formulation is prepared as follow:

Lyophilised MSC exosomes were prepared as previously described in PCT/SG2017/050513/WO/2018/070939.

Disperse lyophilized MSC exosomes (or other lyophilized biological materials) in a plant oil (examples of plant oil are olive oil, palm oil, soy oil, coconut oil etc) by homogenization, sonication or other physical means to form a standard solid-in-oil dispersion¹⁸.

Such suspensions can be stored at room temperature (i.e. <40° C.). Just before use, the suspension is homogenised with an emulsifying agent to form an oil in water emulsion.

The emulsifier can be a natural emulsifying agent (e.g. agar, tragacanth, alginates, gum, pectins, acacia, starch, gelatin, albumin, lecithin, cholesterol), semi-synthetic emulsifying agent (e.g. methylcellulose derivatives such as carboxymethylcellulose, starch derivatives such as octenyl succinate anhydride treated starch also known as OSA-starch or synthetic emulsifying agent (e.g. benzalkonium chloride, benzethonium chloride, sodium oleate potassium oleate, triethanolamine stearate, detergents such as sodium lauryl sulfate, sodium dioctyl sulfosuccinate, sodium docusate), sorbitan esters (Spans) and polyoxyethylene derivatives of sorbitan esters (Tweens), glyceryl esters).

### Example 3. Protocol for Preparing a Formulation

A. Resuspend 500 ug lyophilized exosome in 500 μL olive oil. Store this solid-in-oil suspension at room temperature (i.e. <30° C.).

B. Just before use, mix the solid-in-oil suspension with 2 ml of an emulsifying cream containing 2% (w/v) Seppic plus 400 in water (Seppic 400 is a commercial emulsifying agent consisting of Polyacrylate-13, Polyisobutene & Polysorbate 20 sold by Seppic, https://www.seppic.com) SEPIPLUS™ 400

C. Homogenise well and apply on skin

### Example 4. Stability of Exosome as a Solid in Oil Suspension

Lyophilized exosome was resuspended in olive oil and kept at 40° C. for one week.

The suspension was extracted with water and assayed for CD73 activity. The activity was measured against the CD73 activity in an equivalent amount of lyophilized exosome. The CD73 level was 20% lower, presumably from the loss in extraction and heat. Therefore, if kept at room temperature of ˜30° C., the lyophilized exosome is likely to be stable.

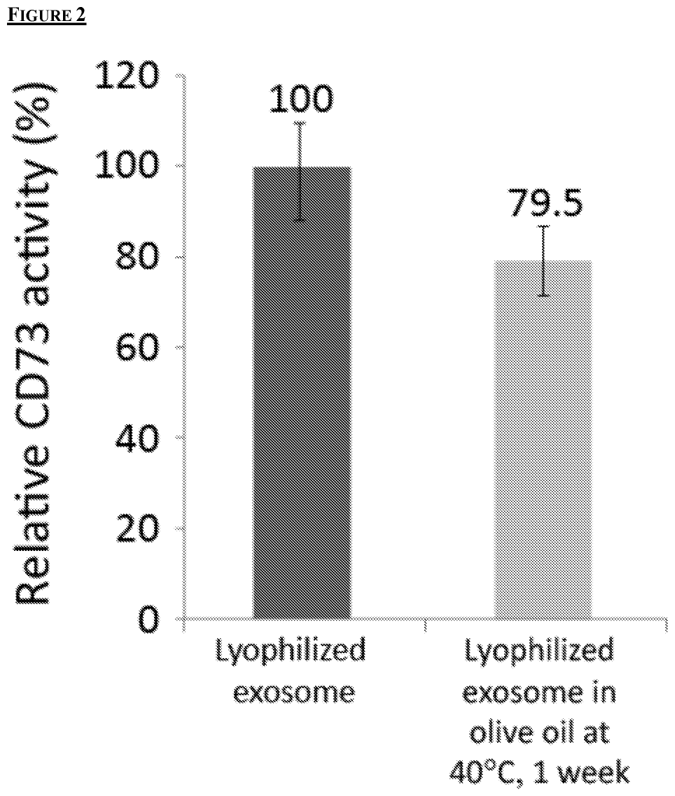

Results are shown in FIG. 2.

Keeping lyophilized exosomes as solid in oil suspension helps stabilize the exosome at room temperature by excluding air and moisture. A further advantage is that this also facilitate the formulation of the exosome into an easily applicable topical cream emulsion. The CD73 was measured as previously described¹⁹.

### Example 5. Alleviating the Immune Reactivity of Psoriasis by Topical Application of Exosomes

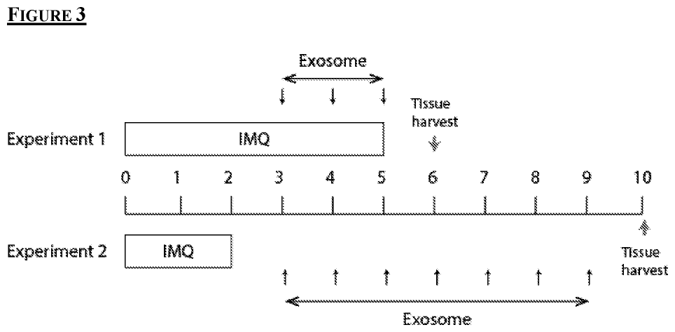

In this study, the efficacy of topically applied exosome or/and YLL liposome cream in alleviating immune reactivity in an imiquimod (IMQ) induced psoriasis-like inflammation model in 6-9 weeks old male Balb/c mice was investigated (FIG. 3).

All mice are kept in quarantine for 3 weeks before starting the experiment. Mice were tagged (left ear) for individual identification and weighed. 1.5 cm×2 cm of the back skin was shaved for IMQ induction. The exosome cream was prepared as above and the control base cream was the exosome cream without the exosomes.

YLL cream was prepared as previously described¹.

### Example 6. Experimental Design: Alleviating the Immune Reactivity of Psoriasis by Topical Application of Exosomes

In Experiment 1, psoriasis was induced by daily topical applications of 50 mg IMQ cream (5% Aldara cream) on the shaved back (on days 0-5). Daily topical application of base, YLL, exosome and YLL+exosome cream was initiated on day 3 for three days (FIG. 3).

Erythema, scaling, and thickness of the back skin were scored independently daily on a scale from 0 to 4. Back skin thickness was measured by electronic calipers as an indicator of edema. The scores were all combined into a cumulative score of 0-12. Spleen was removed from each mouse on day 6 and weighed. Back skin were removed and assayed for Cytokines (TNFα, IL-17 and IL-23) and Membrane Attack Complex C5b-9. In the control arm, mice were treated with just the base cream (vehicle control). Non-IMQ induced skin adjacent to the IMQ induced skin in the control animals were removed to determine baseline cytokine levels in non-IMQ induced skin.

In Experiment 2, psoriasis was induced by daily topical applications of 50 mg IMQ cream (5% Aldara cream) on the shaved back on days 0-2. Daily topical application of base, YLL, exosome and YLL+exosome cream was initiated on day 3 for seven days (FIG. 3).

Erythema, scaling, and thickness of the back skin were scored independently daily on a scale from 0 to 4. Back skin thickness was measured by electronic calipers as an indicator of edema. Spleen was removed from each mouse on day 10 and weighed. Back skin were removed and assayed for Cytokines (TNFα, IL-17 and IL-23) and Terminal Complement Complex C5b-9. In the control arm, mice were treated with just the base cream (vehicle control).

The Cytokines and Terminal Complement Complex C5b-9 were assayed using the following kits, TNFα: R&D system, Cat #: MTA00B, Lot #P175544; IL-17: R&D system, Cat #: M1700, Lot #P163850; IL-23: R&D system, Cat #: M2300, Lot #P173676; MAC (C5b-9): MyBiosource, Cat #:MBS2021342, Lot #L180625054

Statistical analysis was performed using Student's t-test where P values <0.05 was considered as statistically significant.

### Example 7. Results: Skin Phenotype

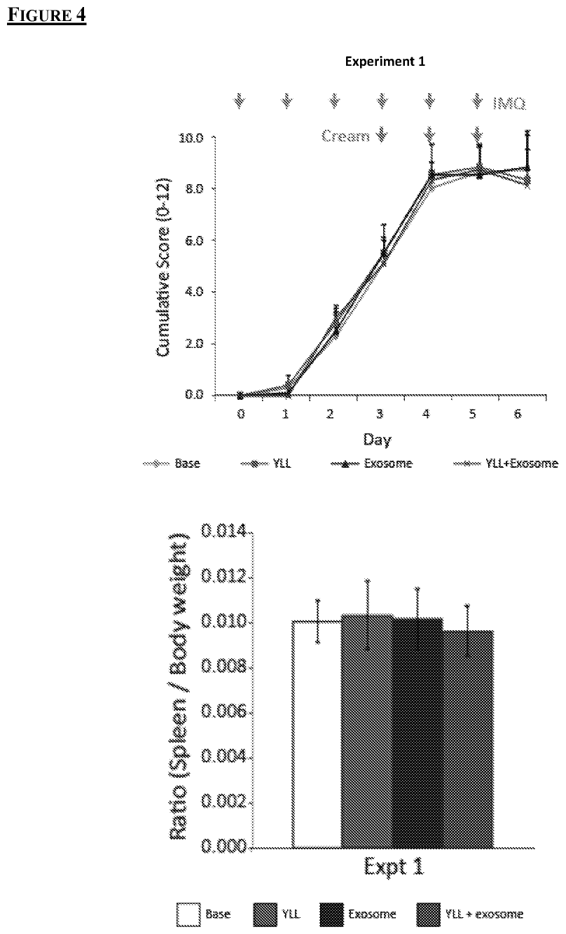

Results are shown in FIG. 4.

As illustrated by Experiments 1 and 2, the cumulative score for skin erythema, scaling and thickness, and spleen weight increased with the duration of IMQ induction, and stopped increasing and then decreased with cessation of IMQ induction.

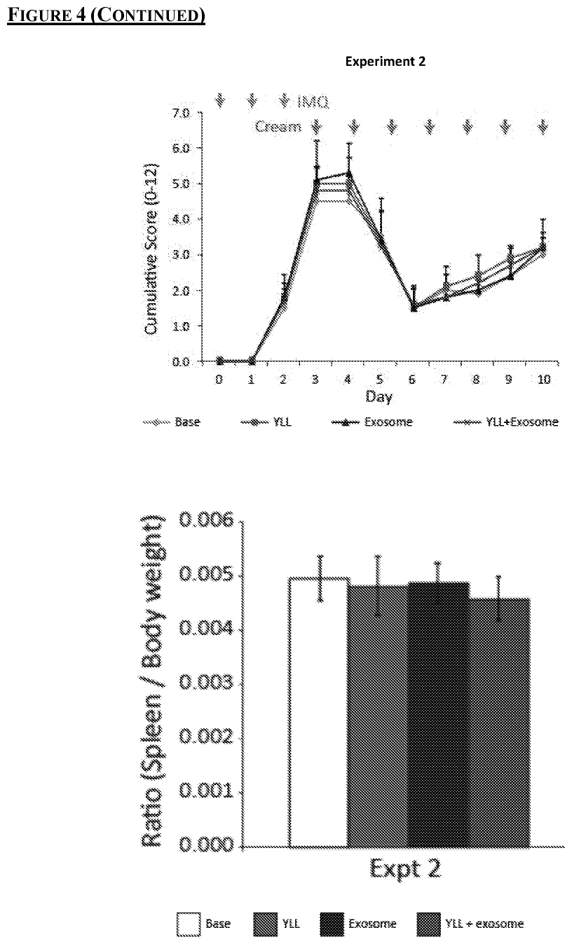

Cream applications in both experiments started on day 3 when the cumulative score is about 5 i.e. similar disease severity. As IMQ induction was extended for another three days in Experiment 1, the cumulative score continued to increase despite the application of the creams. In contrast, the cumulative score in Experiment 2 started decreasing one day after the cessation of IMQ induction. However, the cumulative score in Experiment 2 did not return to baseline. A possible reason is that some of the inflammatory immune molecules induced by IMQ were still elevated (see FIG. 5). The higher cumulative score in Experiment 1 signifying higher inflammation was corroborated by the higher spleen/body weight ratio.

There were no significant differences in cumulative skin scores and spleen/body weight ratio between base cream group (vehicle group) and any of the cream treatment groups based on Student's t-test where P values <0.05 was considered as statistically significant. Therefore, exosome or YLL did not ameliorate skin phenotype or systemic effect on the spleen in an IMQ-induced psoriasis-like inflammation model at the tested dose regimes.

### Example 8. Results: Cytokine Induction and Complement Activation

The back skin of each mouse was removed on day 10 (Experiment 1) or day 6 (Experiment 2), and assayed for TNF alpha, IL17, IL23 and C5b-9 terminal complement complex. The level of cytokine was normalized against the weight of the skin. Statistical significance was determined by Student t test.

Results are shown in FIG. 5.

Based on the response of mice treated with the base cream, we observed that the IMQ elicited a different response profile for each of four moieties assayed.

TNF α response was strongest of the four with a ˜6 fold increase as shown in Experiment 1. It also resolved most rapidly when IMQ induction was stopped by returning to baseline within 6 days after IMQ induction as shown in in Experiment 2.

IL17 increased by ˜3 fold and then decreased to a level ˜2 fold higher than the baseline control (i.e. non-IMQ treated skin) in Experiment 2, IL23 increased by ˜3 fold above baseline in Experiment 1 and remained at this level in Experiment 2.

C5b-9 complex was elevated to 1.5 fold above baseline in Experiment 1 and continued to increase to ˜3.0 fold above baseline in Experiment 2.

These observations suggest that TNF α, IL17, IL23 and C5b-9 complex were induced by IMQ. When IMQ application ceased, both TNF α and IL17 receded towards baseline while IL23 remained elevated eight days after the last IMQ application and C5b-9 complex level increased further.

These observations suggest that TNF α, IL17 and IL23 are more important for the acute phase of psoriasis while IL23 and C5b-9 complex are important for the chronic phase of the disease.

A. TNF α in all treatment groups was elevated above baseline in Experiment 1. The baseline referred to the level in the no IMQ induction skin. Relative to the base cream treatment, TNF α level was reduced in YLL, exosome and YLL+exosome treatment groups but this reduction was statistically significant only for the exosome and YLL+exosome treatment groups. In Experiment 2, TNF α in all treatment groups was similar to baseline level.

B. IL 17 was elevated above baseline in both Experiment 1 and 2 but was generally higher in Experiment 1. There was also no statistically significant difference between the IL 17 levels in base and YLL or exosome cream groups in both Experiment 1 and 2 except for the YLL+liposome treatment group. The reduction of IL 17 level in the YLL+liposome cream group compared to the base cream group was statistically significant.

C. IL 23 was elevated above baseline in Experiment 1 and 2. There were no statistically significant difference in IL 23 level for YLL and exosome cream groups versus the base cream group in both Experiments. For the YLL+exosome groups, the reduction in IL-23 level was statistically significant Experiment 2 but not in Experiment 1.

E. Terminal Complement Complex C5b-9 was elevated above baseline in both Experiments 1 and 2. There were no statistically significant difference between the C5b-9 level in base and any of cream treatment groups for Experiment 1. In Experiment 2, the C5b-9 level in YLL, exosome and YLL+exosome treatment groups relative to the base cream group was reduced and this reduction was statistically significant for all three groups.

### Example 9. Results: Statistical Robustness of Experiment 2

Experiment 2 was repeated using the experimental protocol as described above.

In this new experiment, i.e. Experiment 2A, there were also no significant differences in cumulative skin scores and spleen/body weight ratio between base cream group (vehicle group) and exosome treatment group based on Student's t-test where P values <0.05 was considered as statistically significant.

Therefore, this further confirmed that exosome did not ameliorate skin phenotype or systemic effect on the spleen in an IMQ-induced psoriasis-like inflammation model at the tested dose regimes.

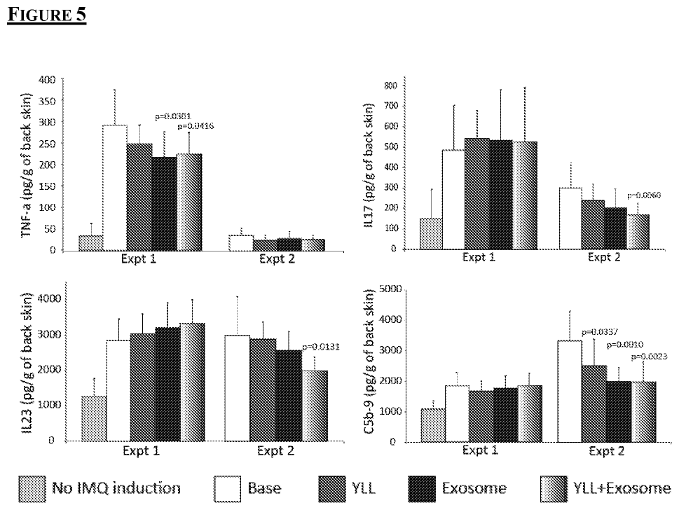

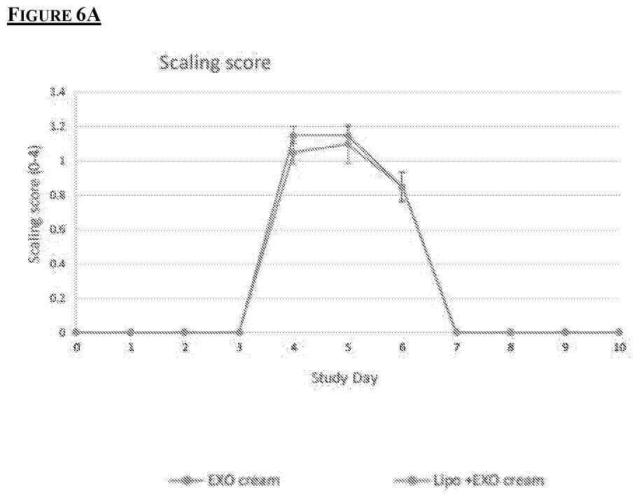

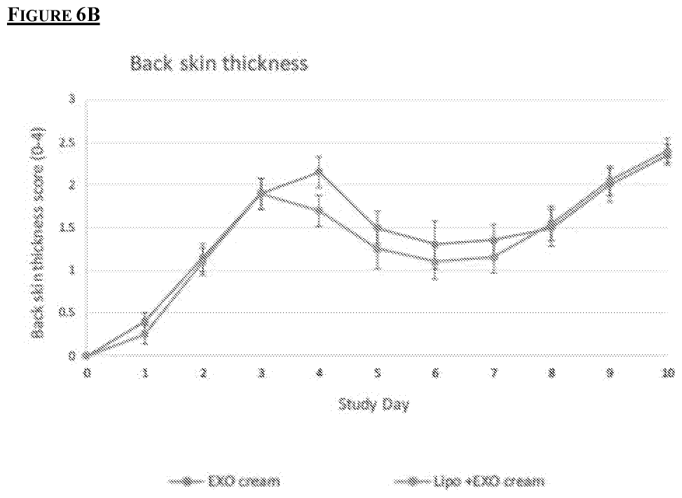

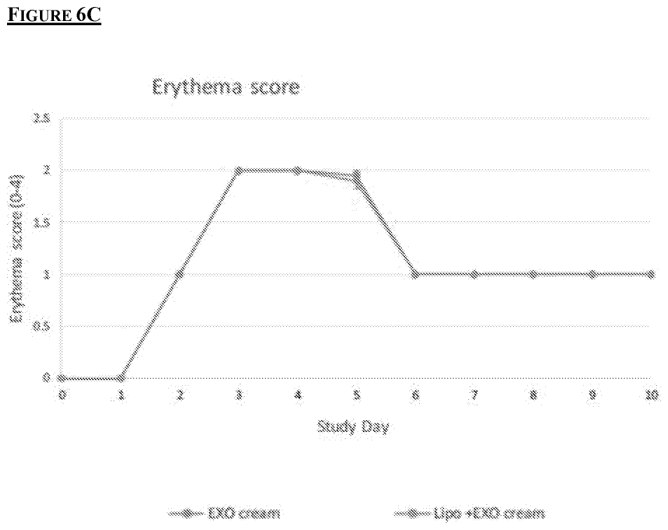

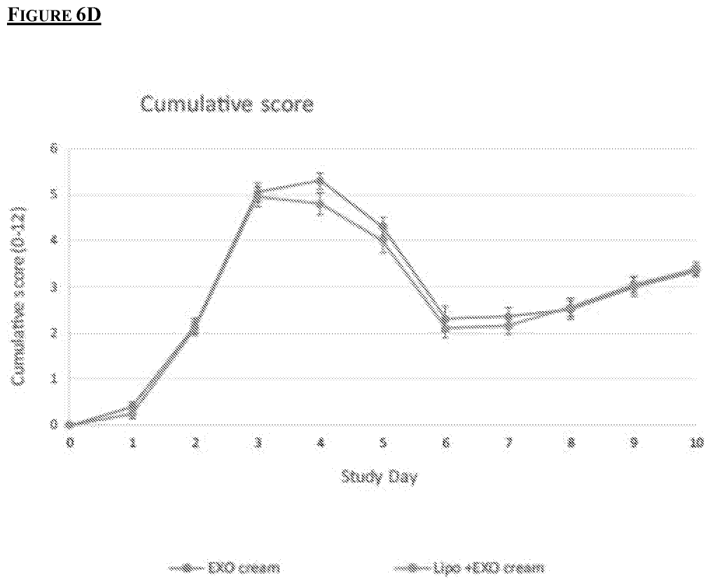

However, there was an improvement in cumulative skin score consisting of scaling, back skin thickness and erythema when exosomes were used in combination with YLL (see FIG. 6A, FIG. 6B, FIG. 6C and FIG. 6D).

The levels of each cytokine and C5b-9 in Experiment 2 and Experiment 2A were normalized to the level of the cytokine in base cream in the respective experiment. The average normalised level of each cytokine and C5b-9 were then analysed.

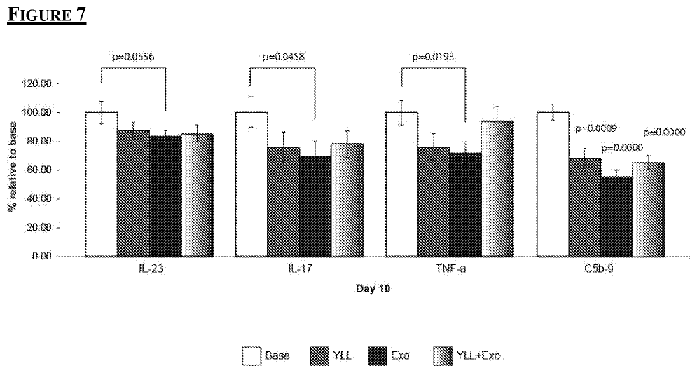

Results are shown in FIG. 7.

All the mouse skins treated with the different exosome cream formulations had reduced levels of IL-23, IL-17, TNF-∞ and C5b-9 relative to that in skin treated with base cream. The reductions in C5b-9 were statistically significant for all formulation groups. The reduction in IL-23, IL-17 and TNF-∞ were statistically close to significant at p=0.056 or significant at p<0.05 for the exosome formulation and not the other formulations.

### Example 10. Discussion

In this study, we demonstrated that topical application of a specially formulated human MSC exosome cream could reduce IL-23, IL-17 and TNF-∞ and Membrane Attack complex C5b-9 in a psoriatic skin lesion in a mouse model of psoriasis.

Unlike our previous observation that YLL has been shown to reduce TNFα in a mouse model of psoriasis in a statistically significant manner³, it has no statistically effect in both Experiment 1 and Experiment 2. This could be due to the difference in experimental design. In the previously reported study which was similar to Experiment 1 except that in the previous study, YLL was applied after each IMQ application. We had attributed the TNFα attenuation to an enhanced skin barrier that prevent infiltration of the chemical irritant³.

The above experiments were designed specifically to test the effects of MSC exosomes on cytokines known to be important in psoriasis, namely TNF α, IL17 and IL23²⁰⁻²². These cytokines are also implicated in other skin disorders such as atopic dermatitis²³, diabetic wound^{24,25}, contact dermatitis²⁶⁻²⁸, acne²⁹, keloids³⁰. Antagonists of TNFα^{22,31}, and more recently, IL17 and IL23³² are used to treat psoriasis. As complement activation is a recent therapeutic target in skin diseases³³ such as psoriasis, lupus erythematosus, cutaneous vasculitis, urticaria, bullous dermatoses and acne³⁴, the effect of exosomes on Membrane Attack complex C5b-9 was also evaluated.

Experiment 1 was designed to determine the effects of exosomes during the acute phase of IMQ irritation while Experiment 2 was designed to determine the effects of exosome post IMQ irritation. As such, the skin in Experiment 1 was in acute inflammation due to active IMQ irritation and this was evident at the time of tissue analysis by the high cumulative psoriatic skin phenotype and the high spleen to body ratio. They were about 2 and 3 times higher than those in Experiment 2 where IMQ application had stopped for eight days at the time of tissue analysis.

The level of TNFα, IL17 and 1L23 was generally higher than those in Experiment 2. This could be due to the shorter exosome treatment but longer IMQ treatment. At 8 days post-IMQ irritation (Experiment 2), TNFα in both base and exosome cream treatment groups was restored to baseline level. IL23 was elevated during IMQ psoriasis induction (Experiment 1) and remained elevated 8 days after a 3 day IMQ psoriasis inducing regime (Experiment 2). Unlike the cytokines, the terminal complement complex C5b-9 was elevated higher in Experiment 2 than 1. Since this occurred eight days after IMQ-irritation ceased and not during IMQ irritation, the increase suggests that the complex may play a role in chronic inflammation that manifests after acute inflammation.

MSC exosomes are known to have potent immunomodulatory properties' and have been reported to alleviate severe immune diseases such as GVHD^{15,2} The effect of MSC exosome in attenuating TNFα is consistent with its anti-inflammatory activities and neutralising effect against inflammatory agents such as LPS and Con A¹ while reduction in the level of terminal complement complex C5b-9 could be attributed to the presence of CD59 on MSC exosomes which has been shown to inhibit MAC formation³⁵.

However, there was no synergistic effect of exosome and YLL in suppressing TNFα, IL17, 1L23 and terminal complement complex C5b-9. Although yolk lecithin is thought to have some mild anti-inflammatory properties³⁶, it has also been shown to have potent immunoactivating properties³⁷ and is the principal ingredient in vaccine adjuvants such as Amphigen and Adjuplex. Nevertheless, this study demonstrated that topical application of MSC exosome and YLL is not more effective than MSC exosome in alleviating inflammatory cytokines in skin immune diseases.

### REFERENCES

1. Bin, Z., et al. Mesenchymal Stem Cells Secrete Immunologically Active Exosomes. Stem cells and development 23, 1233-1244 (2014).
2. Zhang, B., et al. Mesenchymal stromal cell exosome-enhanced regulatory T-cell production through an antigen-presenting cell-mediated pathway. Cytotherapy 20, 687-696 (2018).
3. Lai, R. C., et al. Topical Application of Yolk Lecithin Liposomes Reinforces Skin Barrier Function Against Chemical Agents Such as Psoriasis-inducing IMQ and Alleviates Disease Phenotype. Clin Exp Dermatol Ther CEDT-146. (2018).
4. Gill, H. S. & Prausnitz, M. R. Coated microneedles for transdermal delivery. Journal of controlled release: official journal of the Controlled Release Society 117, 227-237 (2007).
5. Wang, Y., Thakur, R., Fan, Q. & Michniak, B. Transdermal iontophoresis: combination strategies to improve transdermal iontophoretic drug delivery. European Journal of Pharmaceutics and Biopharmaceutics 60, 179-191 (2005).
6. Herwadkar, A., Sachdeva, V., Taylor, L. F., Silver, H. & Banga, A. K. Low frequency sonophoresis mediated transdermal and intradermal delivery of ketoprofen. International Journal of Pharmaceutics 423, 289-296 (2012).
7. Tanner, T. & Marks, R. Delivering drugs by the transdermal route: review and comment. Skin Research and Technology 14, 249-260 (2008).
8. Leite-Silva, V. R., de Almeida, M. M., Fradin, A., Grice, J. E. & Roberts, M. S. Delivery of drugs applied topically to the skin. Expert Review of Dermatology 7, 383-397 (2012).
9. Cevc, G. Lipid vesicles and other colloids as drug carriers on the skin. Advanced Drug Delivery Reviews 56, 675-711 (2004).
10. van Kuijk-Meuwissen, M. E. M. J., Junginger, H. E. & Bouwstra, J. A. Interactions between liposomes and human skin in vitro, a confocal laser scanning microscopy study. Biochimica et Biophysica Acta (BBA)—Biomembranes 1371, 31-39 (1998).
11. Bahia, A. P. C., Azevedo, E. G., Ferreira, L. A. & Frézard, F. New insights into the mode of action of ultradeformable vesicles using calcein as hydrophilic fluorescent marker. European Journal of Pharmaceutical Sciences 39, 90-96 (2010).
12. Maghraby, G. M. E., Williams, A. C. & Barry, B. W. Can drug-bearing liposomes penetrate intact skin? Journal of Pharmacy and Pharmacology 58, 415-429 (2006).
13. Choi, C. K., Zareno, J., Digman, M. A., Gratton, E. & Horwitz, A. R. Cross-correlated fluctuation analysis reveals phosphorylation-regulated paxillin-FAK complexes in nascent adhesions. Biophysical journal 100, 583-592 (2011).
14. Dreier, J., Sorensen, J. A. & Brewer, J. R. Superresolution and Fluorescence Dynamics Evidence Reveal That Intact Liposomes Do Not Cross the Human Skin Barrier. PLOS ONE 11, e0146514 (2016).
15. Kordelas, L., et al. MSC-derived exosomes: a novel tool to treat therapy-refractory graft-versus-host disease. Leukemia 28, 970 (2014).
16. Matejuk, A. Skin Immunity. Archivum immunologiae et therapias experimentalis 66, 45-54 (2018).
17. Cho, B. S., Kim, J. O., Ha, D. H. & Yi, Y. W. Exosomes derived from human adipose tissue-derived mesenchymal stem cells alleviate atopic dermatitis. Stem cell research & therapy 9, 187-187 (2018).
18. Martins, M., Loureiro, A., Azoia, N. G., Silva, C. & Cavaco-Paulo, A. Protein Formulations for Emulsions and Solid-in-Oil Dispersions. Trends in Biotechnology 34, 496-505 (2016).
19. Lai, R., et al. Mesenchymal Stem Cell Exosomes: The Future MSC-Based Therapy? in Mesenchymal Stem Cell Therapy (ed. Chase, L. G. V., Mohan C.) 39-62 (Humana Press, 2013).
20. Boutet, M. A., Nerviani, A., Gallo Afflitto, G. & Pitzalis, C. Role of the IL-23/IL-17 Axis in Psoriasis and Psoriatic Arthritis: The Clinical Importance of Its Divergence in Skin and Joints. Int J Mol Sci 19(2018).
21. Alunno, A., et al. Targeting the IL-23/IL-17 axis for the treatment of psoriasis and psoriatic arthritis. Expert opinion on biological therapy 15, 1727-1737 (2015).
22. Kerdel, F. A. B. M. TNF Inhibitors in Psoriasis: A Review. Seminars in cutaneous medicine and surgery 34, S37-39 (2015).
23. Leonardi, S., et al. Serum interleukin 17, interleukin 23, and interleukin 10 values in children with atopic eczema/dermatitis syndrome (AEDS): association with clinical severity and phenotype. Allergy and asthma proceedings 36, 74-81 (2015).
24. Lee, J., et al. Interleukin-23 regulates interleukin-17 expression in wounds, and its inhibition accelerates diabetic wound healing through the alteration of macrophage polarization. FASEB journal: official publication of the Federation of American Societies for Experimental Biology 32, 2086-2094 (2018).
25. Xu, F., Zhang, C. & Graves, D. T. Abnormal cell responses and role of TNF-alpha in impaired diabetic wound healing. BioMed research international 2013, 754802 (2013).
26. Todberg, T., Zachariae, C., Krustrup, D. & Skov, L. The effect of treatment with anti-interleukin-17 in patients with allergic contact dermatitis. Contact dermatitis 78, 431-432 (2018).
27. Heo, W. I., et al. The role of interleukin-17 in mouse models of atopic dermatitis and contact dermatitis. Clinical and experimental dermatology 40, 665-671 (2015).
28. Griffiths, C. E., Barker, J. N., Kunkel, S. & Nickoloff, B. J. Modulation of leucocyte adhesion molecules, a T-cell chemotaxin (IL-8) and a regulatory cytokine (TNF-alpha) in allergic contact dermatitis (rhus dermatitis). The British journal of dermatology 124, 519-526 (1991).
29. Kelhälä, H.-L., et al. IL-17/Th17 pathway is activated in acne lesions. PLOS ONE 9, e105238-e105238 (2014).
30. Zhang, Q., et al. Tumor-Like Stem Cells Derived from Human Keloid Are Governed by the Inflammatory Niche Driven by IL-17/IL-6 Axis. PLOS ONE 4, e7798 (2009).
31. Yost, J. & Gudjonsson, J. E. The role of TNF inhibitors in psoriasis therapy: new implications for associated comorbidities. F1000 medicine reports 1, 30 (2009).
32. Campa, M., Mansouri, B., Warren, R. & Menter, A. A Review of Biologic Therapies Targeting IL-23 and IL-17 for Use in Moderate-to-Severe Plaque Psoriasis. Dermatology and therapy 6, 1-12 (2015).
33. Giang, J., et al. Complement Activation in Inflammatory Skin Diseases. Frontiers in Immunology 9(2018).
34. Webster, G. F., Leyden, J. J. & Nilsson, U. R. Complement activation in acne vulgaris: consumption of complement by comedones. Infection and immunity 26, 183-186 (1979).
35. Lai, R. C., et al. Mesenchymal Stem Cell Exosomes: The Future MSC-based Therapy? in Mesenchymal Stem Cell Therapy (eds. Chase, L. G. & Vemuri, M. C.) (Humana Press, 2012).
36. Ganley, O. H., Graessle, O. E. & Robinson, H. J. Anti-inflammatory activity on compounds obtained from egg yolk, peanut oil, and soybean lecithin. The Journal of laboratory and clinical medicine 51, 709-714 (1958).
37. Wegmann, F., et al. The Carbomer-Lecithin Adjuvant Adjuplex Has Potent Immunoactivating Properties and Elicits Protective Adaptive Immunity against Influenza Virus Challenge in Mice. Clinical and vaccine immunology: CVI 22, 1004-1012 (2015).

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the claims.

1. A pharmaceutical composition comprising an oil-in-water emulsion of an exosome, such as a mesenchymal stem cell (MSC) exosome, for the treatment or prevention of a dermatological disease or condition.

2. A pharmaceutical composition according to any preceding claim, in which the dermatological disease or condition comprises atopic dermatitis, psoriasis or eczema.

3. A pharmaceutical composition according to any preceding claim, which is capable of reducing or alleviating one or more of erythema, scaling and thickness in the individual as compared to an individual to which the pharmaceutical composition is not applied.

4. A pharmaceutical composition according to any preceding claim, which is capable of reducing the level of an inflammatory cytokine such as TNFα, IL-17, IL-23 and Membrane Attack Complex C5b-9 as compared to an individual to which the pharmaceutical composition is not applied.

5. A pharmaceutical composition according to any preceding claim, in which the pharmaceutical composition comprises a lotion or is to be topically applied to a skin of an individual in need of treatment.

6. A pharmaceutical composition according to any preceding claim, in which the pharmaceutical composition is to be administered 2 to 3 times daily.

7. A pharmaceutical composition according to any preceding claim, in which the pharmaceutical composition alleviates immune reactivity triggered by exposure of the skin of an individual to an irritant such as Imiquimod (IMQ).

8. A pharmaceutical composition according to any preceding claim, further comprising a liposome comprising a lecithin.

9. A pharmaceutical composition according to claim 8, in which the lecithin comprises egg-yolk lecithin or in which the liposome comprising a lecithin comprises a yolk lecithin liposome (YLL).

10. Use of an oil-in-water emulsion of an exosome, such as a mesenchymal stem cell (MSC) exosome, in the preparation of a medicament for the treatment or prevention of a dermatological disease or condition.

11. Use according to claim 10, which comprises a feature set out in any of claims 2 to 9.

12. A method of preparing a pharmaceutical composition for the treatment or prevention of a dermatological condition, the method comprising:
(a) providing an exosome such as an mesenchymal stem cell exosome (MSC); and
(b) homogenising the exosome with an emulsifying agent to form an oil in water emulsion of the exosome.

13. A method according to claim 12, in which the mesenchymal stem cell exosome (MSC) is provided in (a) as a dispersion in an oil, preferably a plant oil such as olive oil.

14. A method according to claim 12 or 13, in which the preparation comprises a period of storage of the MSC dispersion prior to the homogenising step (b).

15. A method according to claim 12, 13 or 14, in which the emulsifying agent is selected from the group consisting of: a natural emulsifying agent, agar, tragacanth, alginates, gum, pectins, acacia, starch, gelatin, albumin, lecithin, cholesterol), a semi-synthetic emulsifying agent, methylcellulose derivatives such as carboxymethylcellulose, starch derivatives such as octenyl succinate anhydride treated starch also known as OSA-starch, a synthetic emulsifying agent, benzalkonium chloride, benzethonium chloride, sodium oleate potassium oleate, triethanolamine stearate, detergents such as sodium lauryl sulfate, sodium dioctyl sulfosuccinate, sodium docusate), sorbitan esters (Spans) and polyoxyethylene derivatives of sorbitan esters (Tweens) and glyceryl esters.

16. A method according to any of claims 12 to 15, in which the emulsifying agent comprises polyacrylate-13, polyisobutene and polysorbate 20, such as 2% (w/v) SEPIPLUS™ 400 in water.

17. A method according to any of claims 12 to 16, further comprising a step of adding a liposome comprising a lecithin, preferably in which the lecithin comprises egg-yolk lecithin or in which the liposome comprising a lecithin comprises a yolk lecithin liposome (YLL), to the pharmaceutical composition.

18. A pharmaceutical composition prepared by a method according to any of claims 12 to 17.

19. A pharmaceutical composition according to claim 18, which comprises a feature set out in any of claims 2 to 9.
